(19) United States
(12) Patent Application Publication
(10) Pub. No.: US 2025/0037879 A1
(43) Pub. Date: Jan. 30, 2025

(54) SYSTEMS AND METHODS FOR MICROBIOME THERAPEUTICS

(71) Applicant: THE REGENTS OF THE UNIVERSITY OF CALIFORNIA, Oakland, CA (US)

(72) Inventors: Mohammad Soheilypour, Danville, CA (US); Mohammad Mofrad, Lafayette, CA (US)

(21) Appl. No.: 18/696,945

(22) PCT Filed: Sep. 29, 2022
(86) PCT No.: PCT/US2022/077238

§ 371 Date: Mar. 28, 2024

Related U.S. Application Data
(60) Provisional application No. 63/261,831, filed on Sep. 29, 2021.

(51) Int. Cl. G16H 50/50; C12Q 1/04; G16H 20/10

(52) U.S. Cl. CPC G16H 50/50; C12Q 1/04; G16H 20/10

(57) ABSTRACT

Methods and systems are provided for design, improvement, optimization, or personalization of microbiome therapeutics for various diseases. In one embodiment, a method comprises identifying a plurality of microbiome features associated with a disease, building a plurality of simulations by modeling the interactions between a plurality of microbial consortia with a plurality of microbiome samples; predicting one or more microbial consortia that improve the plurality of microbiome features associated with a disease from the plurality of simulations; and optimizing composition of one or more microbial consortia that further improve the plurality of microbiome features associated with a disease from the plurality of simulations or personalizing composition of a microbial consortia according to a patient's baseline microbiome to improve the plurality of microbiome features associated with a disease. In this way, effective microbial consortia for a variety of diseases may be reliably predicted in a high-throughput fashion.

### CROSS-REFERENCE TO RELATED APPLICATION

The present application claims priority to and the benefit of U.S. Provisional Patent Application No. 63/261,831, entitled “DESIGN OF MICROBIOME THERAPEUTICS”, filed on Sep. 29, 2021. The entire contents of the above-listed application are hereby fully incorporated by reference herein for all purposes.

### FIELD

The present description relates generally to a computational platform for evaluating interactions between microbial consortia with therapeutic effects and microbiomes.

### GOVERNMENT RIGHTS

This invention was made with Government support under Grant No. 1938257 awarded by the National Science Foundation and Grant No. R43GM130228 awarded by the National Institutes of Health. The Government has certain rights in the invention.

### BACKGROUND

The human gut microbiome, the complex and dynamic community of microorganisms residing in the gastrointestinal tract, has demonstrated correlational and causal relationships with a variety of human diseases, including but not limited to gastrointestinal diseases such as C. difficile infection, ulcerative colitis, Crohn's disease, irritable bowel syndrome, and inflammatory bowel disease, metabolic diseases such as Type 2 Diabetes, allergic diseases such as food allergy and asthma, brain disorders such as hepatic encephalopathy and multiple sclerosis and other diseases such as nonalcoholic fatty liver disease. These findings have led to an increasing interest to modulate the gut microbiome using microbiome therapeutics, i.e. therapeutics comprising living bacteria, as a new generation of therapeutics for difficult-to-treat diseases.

The development of microbiome therapeutics to effectively treat diseases depends on sufficient potency to significantly impact key disease mechanisms. The identification and tailoring of microbial communities that can address the complexities of human disease remains an ongoing challenge in the development of microbiome therapeutics. The field currently lacks a systematic approach to systematically and cost-effectively develop effective microbiome therapeutics. Specifically, the largest barrier to microbiome therapeutic development is the lack of predictive models to translate early-stage research into therapeutic discovery and development. In addition, lack of a systematic approach for development of microbiome therapeutics results in poor patient stratification in clinical trials and ignoring inter-patient variation in therapeutic response.

The high variability of functional and compositional landscape of gut microbial communities across individuals, resulting in patient-to-patient variations in response to treatment strategies, further complicates the development process. Correct dosing can effectively enhance the outcomes of an otherwise ineffective treatment. Therefore, leading researchers have identified that development of therapeutics tailored to an individual's gut microbiota will form the new frontier in the field of precision medicine. Therefore, in some cases, personalization of microbiome therapeutics may be crucial for effective treatment/prevention. However, all existing microbiome therapeutics are developed as one-size-fits-all, primarily because of the lack of a cost-effective approach to personalize such therapeutics.

### SUMMARY

The present disclosure provides the methods for design, improvement, optimization, or personalization of microbiome therapeutics for various diseases. In one embodiment, a method comprises identifying a plurality of microbiome features associated with a disease, building a plurality of simulations by modeling the interactions between a plurality of microbial consortia with a plurality of microbiome samples; predicting one or more microbial consortia that improve the plurality of microbiome features associated with a disease from the plurality of simulations; and optimizing composition of one or more microbial consortia that further improve the plurality of microbiome features associated with a disease from the plurality of simulations or personalizing composition of a microbial consortia according to a patient's baseline microbiome to improve the plurality of microbiome features associated with a disease. In this way, effective microbial consortia for a variety of diseases may be reliably predicted in a high-throughput fashion.

It should be understood that the brief description above is provided to introduce in a simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a block diagram illustrating an example computing system providing a computational platform for design of microbiome therapeutics for various diseases, according to an embodiment;

FIG. 2 shows a block diagram illustrating an example structure for enhanced design of microbiome therapeutics, including strain selection and consortia optimization, in accordance with certain embodiments of the disclosed technology;

FIG. 3 shows a block diagram illustrating an example module architecture for the microbiome therapeutic modeling platform for predicting individual-specific effect of microbiome therapeutics on the gut microbiome in accordance with certain embodiments of the disclosed technology;

FIG. 4 shows a high-level flow chart illustrating an example method for processing omics data to identify microbial species and reconstruct metabolic models, according to an embodiment;

FIG. 5 shows a high-level flow chart illustrating an example method to identify microbiome features;

FIG. 6 illustrates an example of a model architecture of a neural network in accordance with certain embodiments of the disclosed technology;

FIG. 7 illustrates another example of a model architecture of a neural network in accordance with certain embodiments of the disclosed technology;

FIG. 8 illustrates a pseudo-code for an example optimization algorithm in accordance with certain embodiments;

FIG. 9 shows a block diagram illustrating an example structure for personalization of microbiome therapeutics in accordance with certain embodiments of the disclosed technology;

FIG. 10 shows a set of graphs illustrating that gut microbiome simulations with the computational platform are stable over twenty-four hours according to different metrics including Shannon diversity index and Aitchison distance;

FIG. 11 shows a set of graphs illustrating that the computational platform accurately predicts microbiome dynamics in the absence or presence of microbiome therapeutics according to different methods including Principal Coordinate Analysis (PCoA) of microbial compositions and Aitchison distance; and

FIG. 12 shows a set of graphs illustrating that the computational platform designs microbiome therapeutics with enhanced therapeutic efficacy according to microbiome features and clinical endpoints.

### DETAILED DESCRIPTION

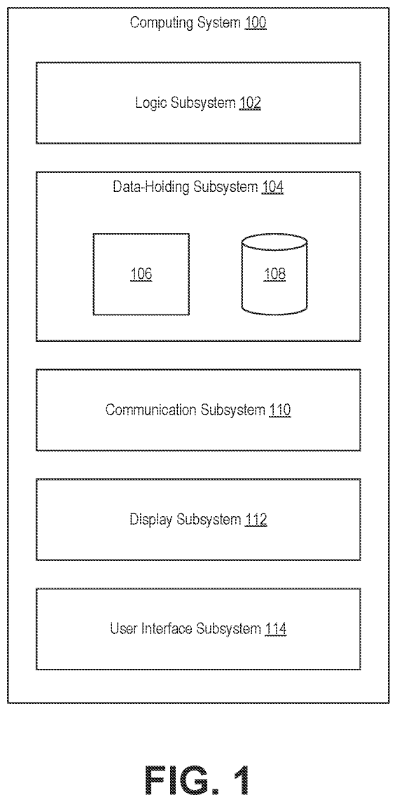

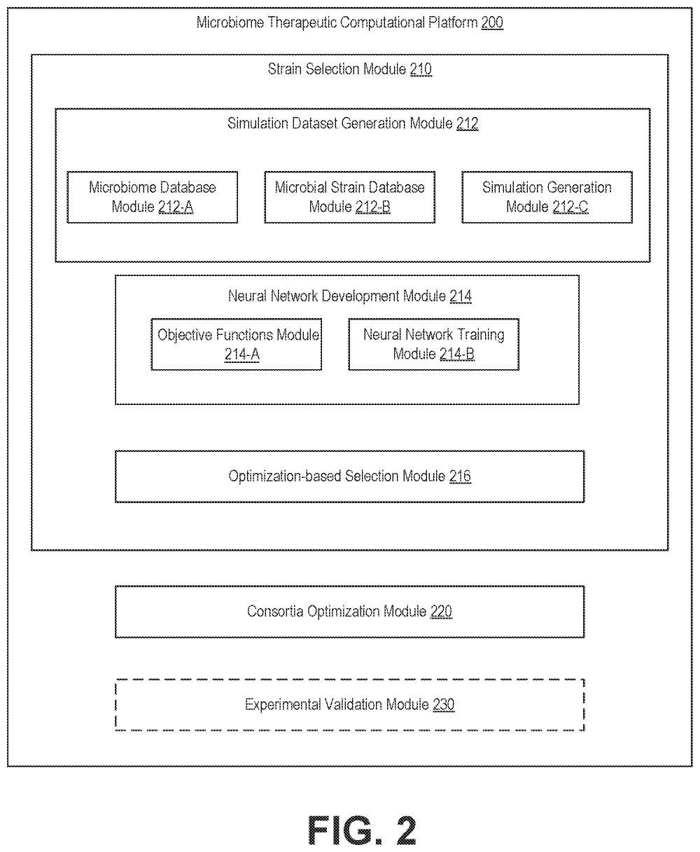

The following description relates to a computational platform for design, improvement, optimization, or personalization of microbiome therapeutics. A computing system, such as the computing system shown in FIG. 1, may provide a computational platform, such as the computational platform shown in FIG. 2, configure to perform high-throughput identification of therapeutic microbial consortia. In particular, the platform enables accurate testing of thousands of microbiome therapeutics, including but not limited to gut microbiome therapeutics, against hundreds of microbiome samples. The platform further enables prediction of interactions between microbiome therapeutics and microbiomes and their effect on the microbiome and host. Further still, the platform incorporates inter-individual variability to explore the mechanistic link between microbial consortia and their associated microbiome-engineering capacity.

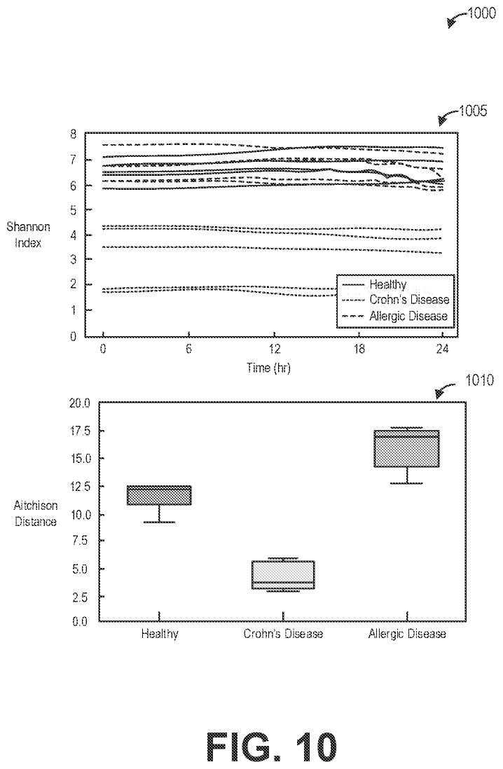

The methods for the computational platform, as shown in FIGS. 2-9, integrate three-dimensional modeling methods, neural networks, and highly efficient optimization algorithms to achieve accurate identification of therapeutic microbial consortia. Additionally, these methods further improve or optimize efficacy of identified microbiome therapeutics and enables the identification of responder and non-responder patient populations to a microbiome therapeutic. The systems and methods provided herein thus capture the individual-specific composition and functional landscape of the human gut microbiome in interaction with microbiome therapeutics, enable the cost-effective and accurate design of the microbiome therapeutics, and are experimentally validated on multiple levels for reliable predictions. Comparisons of the simulated or in silico experiments against more traditional in vitro and in vivo experiments, as shown in FIGS. 10-12, demonstrate that the systems and methods provided herein achieve highly accurate predictions to design highly effective microbiome therapeutics.

Turning now to the drawings, FIG. 1 shows a block diagram illustrating an example computing system 100 providing a computational platform for enhanced design of microbiome therapeutics for various diseases. It should be appreciated that the architecture of the computing system 100 is exemplary and non-limiting, and that other computer architectures may be used for a computing device without departing from the scope of the present disclosure. In different embodiments, the computing system 100 may comprise a mainframe computer, a server computer, a desktop computer, a laptop computer, a tablet computer, a network computing device, a mobile computing device, a mobile communication device, and so on. As depicted, the computing system 100 comprises a logic subsystem 102 and a data-holding subsystem 104. The computing system 100 may further include a communication subsystem 110, a display subsystem 112, and a user interface subsystem 114.

The logic subsystem 102 may include one or more physical devices configured to execute one or more instructions. For example, the logic subsystem 102 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem 102 may include one or more processors that are configured to execute software instructions. In some examples, the logic subsystem 102 may include one or more hardware and/or firmware logic machines configured to execute hardware and/or firmware instructions. Processors of the logic subsystem 102 may be single core or multi-core, and the programs executed thereon may be configured for parallel or distributed processing. The logic subsystem 102 may optionally include individual components that are distributed throughout two or more devices, which may be remotely located and/or configured for coordinated processing. One or more aspects of the logic subsystem 102 may be virtualized and executed by remotely accessible networked computing devices configured in a cloud computing configuration.

The data-holding subsystem 104 may include one or more physical, non-transitory devices configured to hold data and/or instructions executable by the logic subsystem 102 to implement the herein-described methods and processes. When such methods and processes are implemented, the state of data-holding subsystem may be transformed (for example, to hold different data).

The data-holding subsystem 104 may include removable media and/or built-in devices. Data-holding subsystem 104 may include optical memory (for example, CD, DVD, HD-DVD, Blu-Ray Disc, and so on), and/or magnetic memory devices (for example, hard disk drive, floppy disk drive, tape drive, MRAM, and so on), and the like. The data-holding subsystem 104 may include devices with one or more of the following characteristics: volatile, nonvolatile, dynamic, static, read/write, read-only, random access, sequential access, location addressable, file addressable, and content addressable. In some embodiments, the logic subsystem 102 and the data-holding subsystem 104 may be integrated into one or more common devices, such as an application specific integrated circuit or a system on a chip. In other embodiments, the data-holding subsystem 104 may include individual components that are distributed throughout two or more devices, which may be remotely located and accessible through a networked configuration.

When included, the communication subsystem 110 may be configured to communicatively couple the computing system 100 with one or more other computing devices. The communication subsystem 110 may include wired and/or wireless communication devices compatible with one or more different communication protocols. As non-limiting examples, the communication subsystem 110 may be configured for communication via a wireless telephone network, a wireless local area network, a wired local area network, a wireless wide area network, a wired wide area network, and so on. In some examples, the communications subsystem 110 may enable the computing system 100 to send and/or receive messages to and/or from other computing systems via a network such as the public Internet.

When included, the display subsystem 112 may be used to present a visual representation of data held by data-holding subsystem 104. As the herein-described methods and processes change the data held by the data-holding subsystem 104, and thus transform the state of the data-holding subsystem 104, the state of display subsystem 112 may likewise be transformed to visually represent changes in the underlying data. The display subsystem 112 may include one or more display devices utilizing any type of display technology. Such display devices may be combined with the logic subsystem 102 and/or the data-holding subsystem 104 in a shared enclosure, or such display devices may comprise peripheral display devices.

When included, the user interface subsystem 114 may include one or more physical devices configured to facilitate interactions between a user and the computing system 100. For example, the user interface subsystem 114 may comprise one or more user input devices including but not limited to a keyboard, a mouse, a camera, a microphone, a touch screen, and so on.

As described further herein, the computing system 100 provides a computational platform for enhanced design of microbiome therapeutics for various diseases. To that end, the data-holding subsystem 104 may store a computational platform 106 for enhanced design of microbiome therapeutics for various diseases. An example computational platform 106 is described further herein with regard to FIG. 2. The data-holding subsystem 104 may further store one or more databases 108, including one or more of a database of gut microbial strains such as the Unified Human Gastrointestinal Genome (UHGG) collection, a database of omics data from various microbiome samples, a database of simulated microbiome samples in interaction with various microbiome therapeutics, and so on.

FIG. 2 shows a block diagram illustrating an example module architecture for a microbiome therapeutic computational platform 200 for design of microbiome therapeutics for various diseases, according to an embodiment. The computational platform 200 may be implemented as the computational platform 106 in the computing system 100, as an illustrative and non-limiting example. It should be appreciated that the modules of the computational platform 200 are exemplary and non-limiting, and that the computational platform 200 may be implemented with other modules and sub-modules without departing from the scope of the present disclosure.

The computational platform 200 comprises a plurality of modules, including a strain selection module 210 configured to predict one or more microbial consortia that may have therapeutic effects for a certain diseases and a consortia optimization module 220 configured to optimize the abundance levels of the predicted microbial consortia to improve their therapeutic effects, and optionally an experimental validation module 230 configured to validate modules of the computing platform 200 based on experimental data.

The strain selection module 210 may comprise a simulation dataset generation module 212 configured to build a dataset of simulations for strain selection. This data may be generated by simulating the interactions between a variety of random microbial consortia with a variety of microbiome samples. Various data types may be collected from these simulations and may be stored in data-holding subsystem 104 for future use. As illustrative and non-limiting examples, abundance of microbial taxa and concentration of different metabolites during or at the end of simulations may be stored.

The simulation dataset generation module may comprise a microbiome database module 212-A configured to store omics data, including one or more of metagenomic, metatranscriptomic, metaproteomic, or metabolomic data, from a set of microbiome samples from patients with a certain disease. As an illustrative and non-limiting example, the microbiome database module could include at least 100 metagenomic samples from patients with Type 2 Diabetes to capture a wide microbiome inter-individual variability.

The simulation dataset generation module may further comprise a microbial strain database module 212-B configured to store a database of microbial strains that could be used to develop microbiome therapeutics. These microbial strains may be collected and stored in house or may be retrieved from a variety of microbiome databases such as the Human Gut MAG dataset, the Unified Human Gastrointestinal Genome Collection, and the Human Reference Gut Microbiome, as illustrative and non-limiting examples. As an example, complete high-quality genomes (≤5% contamination) from each database may be retrieved, combined, and redundant genomes may be removed, to form a microbial strain database with at least 1,000 microbial strains with potential therapeutic efficacy.

The simulation dataset generation module may further comprise a simulation generation module 212-C configured to generate simulations of a plurality of microbiome samples stored in microbiome database module 212-A in interaction with a plurality of microbial consortia obtained from the microbial strain database module 212-B for a certain amount of simulation time. The simulations may be conducted using the microbiome therapeutic modeling platform 300. As an illustrative and non-limiting example, 5000 microbial consortia from the microbial strain database module 212-B and 100 microbiome samples from the microbiome database module 212-A may be used to form 500,000 simulations. Alternatively, as an illustrative and non-limiting example, microbiome samples may be clustered according to their genomic content using Principal Component Analysis (PCoA) to identify closely-related samples and then a representative microbiome sample may be selected for each cluster for simulations with microbial consortia.

The strain selection module 210 may further optionally comprise a neural network development module 214 configured to train one or more neural networks to predict final microbiome composition or certain microbiome features based on initial microbiome composition interacting with microbial consortia using the data generated in simulation dataset generation module 212. Use of neural networks may enable a wider search for potential microbial consortia with therapeutic effects for target diseases.

It should be appreciated that neural network development module 214 may be replaced with a machine learning development module configured to train one or more machine learning models to predict final microbiome composition or certain microbiome features based on initial microbiome composition interacting with microbial consortia using the data generated in simulation dataset generation module 212 without departing from the scope of the present disclosure.

The neural network development module 214 may comprise an objective functions module 214-A configured to store, identify, or characterize certain microbiome features associated with target patient population. As illustrative and non-limiting examples, microbiome features, including but not limited to taxonomic features or metabolic features, may be previously known or identified such as microbial diversity, abundance of microbial taxa, or concentration of certain metabolites, as illustrative and non-limiting examples.

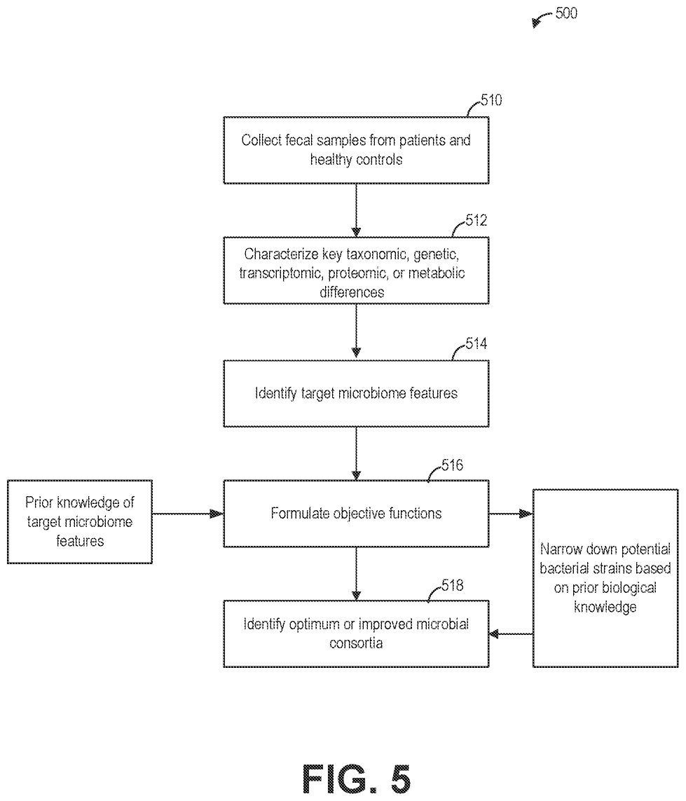

As an illustrative and non-limiting example, FIG. 5 shows a high-level flowchart illustrating an example method to identify microbiome features. In the case of unknown microbiome features, they may be identified, as an illustrative and non-limiting example, by first collecting and/or retrieving fecal samples from patients with the target indication as well as healthy control individuals, as indicated at 510, and then characterizing key taxonomic, genetic, transcriptomic, proteomic, or metabolic differences between the two cohorts, as indicated at 512, using metagenomic, metatranscriptomic, metaproteomic, metabolomic sequencing or any combination thereof.

Microbiome features may be identified, as indicated by 514, by a variety of methods including but not limited to comparing the abundance of microorganisms at phylum, class, order, family, genus, species, or strain level in healthy individuals versus patients and identify phyla, classes, orders, families, genera, species, or strains that are statistically significantly higher or lower in abundance in patients over healthy individuals, comparing concentration of metabolites or classes of metabolites in healthy individuals versus patients to identify metabolites that are statistically significantly higher or lower in concentration in patients over healthy individuals, using bioinformatics and/or machine learning methods to identify microbiome features, including but not limited to microorganisms at any taxonomic level, metabolite concentrations, diversity metrics such as alpha or beta diversity, microbial gene content, microbial gene products, or microbial pathways or any combination thereof that are significantly correlated with endpoints of interest for the target indication.

It should be appreciated that host features and/or clinical endpoints may be used in combination and/or instead of microbiome features without departing from the scope of the present disclosure. As an illustrative and non-limiting example, area under the curve (AUC) of core body temperature may be used as a clinical endpoint. As another example, host's metabolic and/or immune profile may be used as a host feature. As another example, plasma levels of certain metabolites could be used as a clinical endpoint. Host features may be characterized using genomics, transcriptomics, proteomics, metabolomics, or any combination thereof.

Additionally, in case prior knowledge of potential bacterial strains for improving the target diseases may be available, the landscape of bacterial strains to be explored by the algorithm may be narrowed down accordingly.

As an illustrative and non-limiting example, ammonia production in the gut microbiome may be used as a microbiome feature in hepatic encephalopathy. As another example, production of tauroursodeoxycholic acid (TUDCA) may be used as a microbiome feature for diabetic retinopathy. As another example, Akkermansia muciniphila, Ruthenibacterium lactatiformans, Hungatella hathewayi, Eisenberqiella tayi Faecalibacterium prausnitzii and Blautia species may be used as microbiome features for multiple sclerosis.

The objective functions module 214-A may convert microbiome composition or microbial features to mathematical equations that may be used for training and validation of one or more neural networks. As an example, high abundances of some bacterial genera such as Bacteroides, Akkermansia, Faecalibacterium, Roseburia, and Bifidobacterium, as illustrative and non-limiting examples, (Group I) may be negatively associated with a disease, while abundance of other bacterial genera such as Fusobacterium and Ruminococcus, as illustrative and non-limiting examples, (Group II) may be positively associated with a certain disease.

Therefore, a higher abundance of Group I and a lower burden of Group II may be desired. Therefore, the loss functions for training of one or more neural networks may be mean absolute error (MAE) for (1) relative abundance of Group I bacterial genera, and (2) relative abundance of Group II bacterial genera. Sₜₒₜ may be the sum of relative abundances of Bacteroides, Akkermansia, Faecalibacterium, Roseburia, and Bifidobacterium, [symbol] the sum of predicted relative abundances of these genera, Fₜₒₜ may be the sum of relative abundances of Fusobacterium and Ruminococcus, and [symbol] the sum of predicted relative abundances of these microbes. Therefore, Group I and Group II loss functions may be defined according to:

$${S}_{loss} = \frac{1}{n} {\sum }_{i = 1}^{n} {t}_{} - {S}_{tot} \vert$$

$${F}_{loss} = \frac{1}{n} {\sum }_{i = 1}^{n} {t}_{} - {F}_{tot} \vert$$

The neural network development module may further comprise a neural network training module 214-B that may train and validate one or more neural networks. The input to these neural networks may be the composition of microbiome samples used in simulation dataset generation module 212 and the output may be microbiome composition at intervals or the end of each simulation or microbiome features as described above.

As an illustrative and non-limiting example, the neural network may comprise a fully connected deep learning model. An 80-20 split in the training dataset may be used. All the inputs and outputs may be normalized from zero to one to ensure quicker convergence. Therefore, relative abundance for all the microbial strains in the target microbiota as well as the therapeutic candidates may be used. The minimum and maximum will be calculated based on the training set and those values will be used to normalize both the training and testing set. Tensorflow and Keras may be used to create the model architecture. A rectified linear unit (ReLU) for activation functions may be used, and dropout layers may be placed directly after any fully connected layers to prevent overfitting. Dropout layers may be at uniform intervals between 0.1 and 0.5. The Adam optimizer may be used to speed up the convergence of the models.

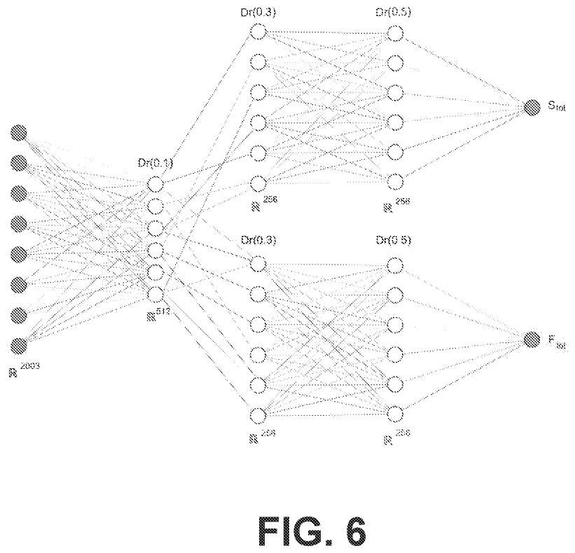

As an illustrative and non-limiting example, FIG. 6 illustrates a model architecture that may involve three sets of a fully connected layer followed by a dropout layer, where, after the first fully connected and dropout layer, the model may bifurcate into two branches: one for Sₜₒₜ and one for Fₜₒₜ.

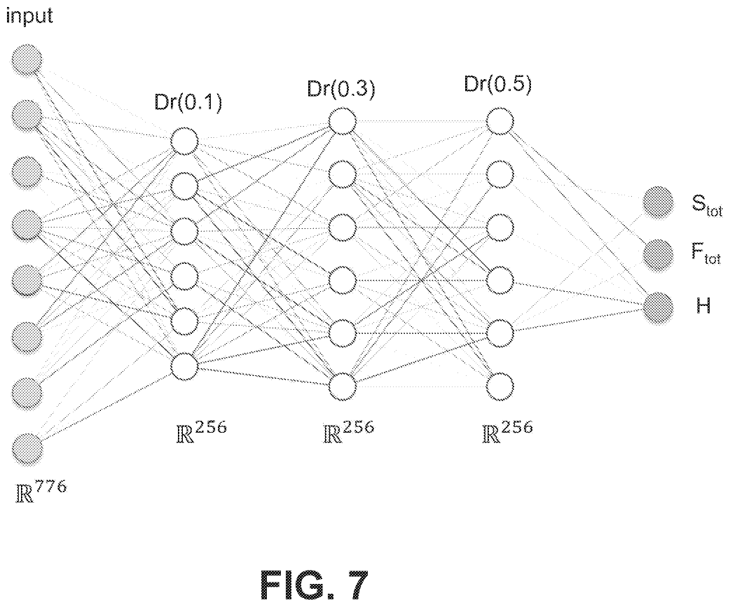

As an illustrative and non-limiting example, FIG. 7 illustrates a model architecture that may involve three sets of a fully connected layer followed by a dropout layer, and the output layer comprises three nodes: one for Sₜₒₜ, one for Fₜₒₜ, and one of another parameter such as alpha diversity (Shannon index) H.

The strain selection module 210 may further comprise an optimization-based selection module 216 configured to search and find microbial consortia with therapeutic potential for a certain disease according to objective functions defined in objective functions module 214-A and using the simulations generated in simulation generation module 212-C.

Alternatively, the optimization-based selection module 216 may use one or more neural networks developed in the neural network development module 214. This module may identify optimum microbial strains according to target objective functions. Development or optimization or improvement of MTs may be a single-objective or multi-objective optimization problem, as there are potentially multiple factors that identify the efficacy of treatment. In a multi-objective problem, there is usually no single “best” point in the solution space that surpasses all other points with respect to all objectives.

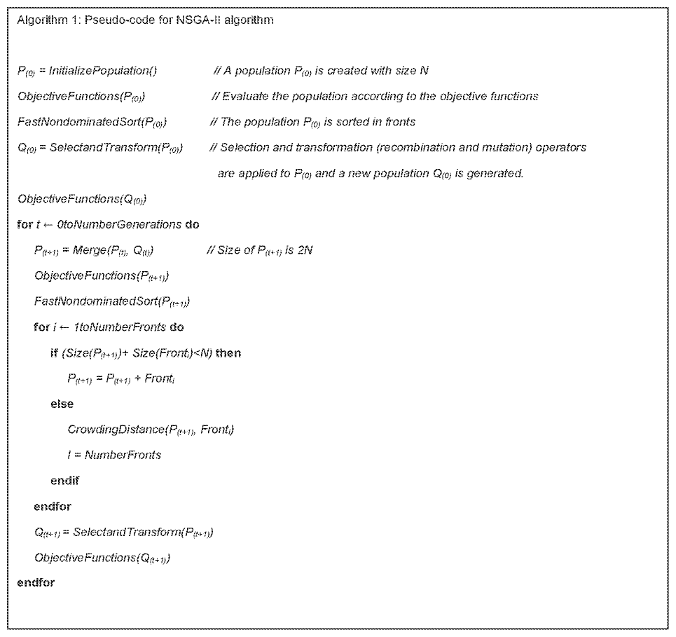

Therefore, multi-objective improvement or optimization methods provide non-dominated or Pareto-optimal solution sets, i.e. solutions in which none of the objective functions can be improved without degrading some of the other objective functions. Pareto solutions are classified into fronts, with the first front being the solutions that are not dominated by any other solution, the second front being the solutions only dominated by the first front, and so on. After the number of iterations is fulfilled, the first Pareto front is the optimized set of solutions. As an illustrative and non-limiting example, FIG. 8 illustrates a pseudo-code for a multi-objective optimization algorithm, i.e. NSGA-II.

The consortia optimization module 220 is configured to improve strain cell count, relative abundance, or absolute abundance for microbial consortia identified by strain selection module 210 for enhanced therapeutic outcomes. In strain selection module 210, a nominal cell count, relative abundance, or absolute abundance may be used for all the strains. In the consortia optimization module 220, the cell counts or relative abundances may be improved to according to objective functions determined in objective functions module 214-A using a single-objective or multi-objective optimization algorithm.

As an illustrative and non-limiting example, NSGA-II algorithm may be used, which is shown in FIG. 8. This algorithm initiates the improvement or optimization process with a random population of potential solutions, with each solution containing values for the cell count, relative abundance, or absolute abundance of each of the potential bacterial strains in a microbial consortium. The objective functions may be evaluated for all members of this initial population using the microbiome therapeutic modeling platform 300.

The algorithm may evaluate the associated values for objective functions, such as maximize(Sₜₒₜ) and minimize(Fₜₒₜ), as illustrative and non-limiting examples, for the initial population of solutions. Subsequently, these solutions may be ranked, followed by a selection and transformation procedure, which creates another set of potential solutions. The two solution sets may then be combined.

This process may be repeated for the number of desired generations until the algorithm converges and provides the final set of improved or optimized composition of microbial consortia. The initial population size (N), number of generations, and recombination and mutation rates may be adjusted during model calibration.

Cell count for the strains may be limited, for example due to manufacturing limitations, which may limit the search landscape for the optimization algorithm. However, the optimization algorithm would be applicable to any cell count, no matter how small or large.

The experimental validation module 230 is configured to use experimental data to validate the computational platform 200. For example, to validate the platform in vitro experiments, in vivo animal models, or human trials may be employed.

As an illustrative and non-limiting example, the experimental validation module 230 may validate microbiome composition or microbiome features over time. In one example, the experimental validation module 230 may use experimental data obtained via ex vivo cultures of fecal samples and microbiome therapeutics including consortia predicted by the strain selection module 210 and optimized by the consortia optimization module 220. After pre-processing, a glycerol stock of the fecal sample may be used to inoculate in modified Gifu Anaerobic Medium (mGAM) broth, or modified Gifu Anaerobic Medium (mGAM) broth or Gut Microbiota Medium (GMM), in order to grow for 48 hours. After the growth period, an aliquot of the culture may be used for interaction with candidate microbiome therapeutics in the media. Cultures may be incubated for 24 hours in an anaerobic chamber.

Experiments may be performed in triplicate to obtain statistically significant and reproducible results. Samples may then be collected and centrifuged to remove any fecal bacteria, at multiple data points such as 0, 1 h, 2 h, 6 h, 8 h, 12 h, 16 h and 24 hours and may then be analyzed using sequencing or HPLC-MS.

As another illustrative and non-limiting example, the predicted improved or optimum combinations of bacterial strains could be administered in vivo. Gut microbiome structure could be profiled at baseline, during treatment, and at study termination to evaluate the influence of the improved or optimum microbiome therapeutic candidates on the distinct microbiota in situ. For example, target microbiome features may be evaluated along with overall total community diversity. The success criteria for this validation may be defined as, for instance, achieving ≤15% error for alpha diversity prediction and ≤10% error for prediction of Sₜₒₜ and Fₜₒₜ. Quantitative comparison of microbiome features such as microbiota structure as well as host features and clinical endpoints may be used to compare the efficacy of the improved or optimized treatments.

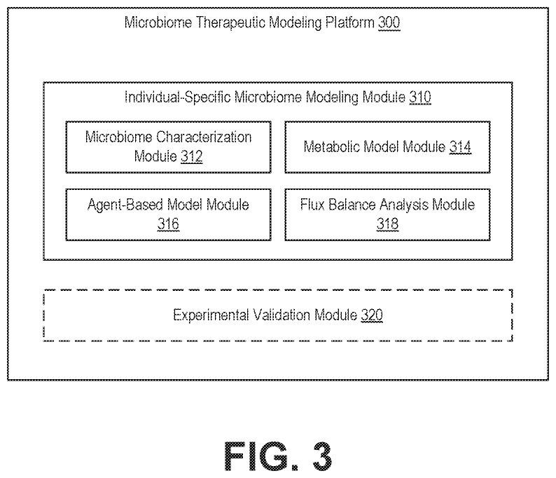

FIG. 3 shows a block diagram illustrating an example module architecture for a microbiome therapeutic modeling platform 300 for predicting individual-specific effect of microbiome therapeutics on the gut microbiome, according to an embodiment. It should be appreciated that the modules of the computational platform 300 are exemplary and non-limiting, and that the computational platform 300 may be implemented with other modules and sub-modules without departing from the scope of the present disclosure.

The computational platform 300 comprises a plurality of modules, including an individual-specific microbiome modeling module 310 configured to simulate interaction of the target microbiome therapeutic with an in silico gut microbiome, and optionally an experimental validation module 320 configured to validate modules of the computing platform 300 based on experimental data.

The individual-specific microbiome modeling module 310 comprises a microbiome characterization module 312, a metabolic model module 314, an agent-based model module 316, and a flux balance analysis module 318. The microbiome characterization module 312 may extract types and abundance of microbial species from omics data.

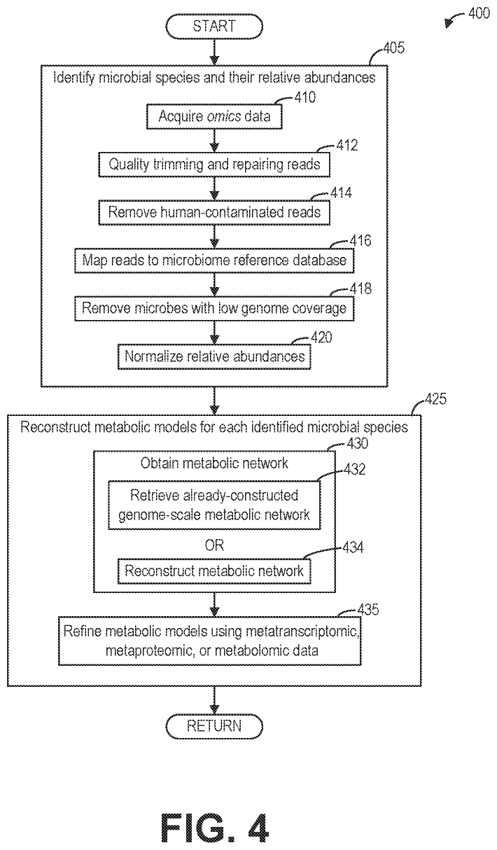

For example, FIG. 4 shows an example method 400 for the microbiome characterization module 312. Method 400 begins at 405, where method 400 identifies microbial species and their relative abundances. To that end, method 400 obtains raw 16S rRNA or metagenomic data either through 16S rRNA or shotgun sequencing of the target microbiome or via the NCBI sequence read archive (SRA).

At 412, method 400 quality trims the reads using Trimmomatic and then re-pairs the reads using the BBmap repair tool. At 414, method 400 removes human contaminant sequences by mapping the paired reads to human reference genome build 38 (GRCh38) using Burrows-Wheeler Aligner (BWA). Cross-mapped reads (reads mapped to multiple positions) may be filtered out by discarding mapped reads with a low-quality score using SAMtools. At 416, method 400 then maps the pre-processed reads to a reference gut microbiome database.

At 418, method 400 removes microbes with low genome coverage. For example, the abundance of each microbial species may be calculated by adding up the sequence length of reads mapped to a unique region of a species' genome, normalized by the total size of the species' genome. A minimum genome coverage (for example, 1%) may be assigned for each identified microorganism to reduce the number of false positives. At 420, the resulting coverages for each microorganism may be normalized to 1 Gb to obtain relative microbe abundances.

After identifying the microbial species and their relative abundances at 405, method 400 proceeds to 425, where method 400 reconstructs metabolic models for each identified microbial species. Genome-scale metabolic models relate metabolic genes with metabolic pathways. Thus, at 430, the metabolic model module 314 retrieves or reconstructs the metabolic models associated with the microorganisms identified by microbiome characterization module 312.

The metabolic model module 312 may use metabolic model datasets at 432, in some examples, or in other examples the metabolic model module 312 may, at 434, use metabolic network reconstruction methods or tools, such as the CarveMe tool to build metabolic models using reference genomes. After obtaining the metabolic networks, method 400 continues to 435.

At 435, method 400 further refines the metabolic models using metatranscriptomic, metaproteomic, or metabolomic data. First, gene or protein expression data is binarized into on and off states. Subsequently, these states are used to modify metabolic pathways by mapping to corresponding genome-scale metabolic network reconstructions. The reconstructed metabolic models may be associated with a corresponding agent type in the agent-based model(s) module 246.

Referring again to FIG. 3, the agent-based model(s) module 316 constructs an individual-specific model of the target gut microbiome in interaction with the target microbiome therapeutic. The primary inputs to the model may be microbial species identified in microbiome characterization module 312, relative abundance of each microorganism identified in microbiome characterization module 312, metabolic networks associated with each microorganism identified in metabolic model(s) module 314, and metabolites that should be present to support these metabolic pathways. Microbiome therapeutics may be added to the system similar to host bacteria and their metabolic models are integrated with the rest of the bacteria in the system.

Additional inputs to the model may include simulation parameters such as the size of the system (e.g., in micrometers), the time step (e.g., in seconds), and the number of desired simulation steps as well as molecular fields in the system, their diffusion coefficients, and their initial concentrations. The agent-based model(s) module 246 may then construct the three-dimensional environment of the simulation where agents (representing microorganisms) are distributed randomly, with each microbe given random initial biomass according to a median cell dry weight (e.g. 0.489 pg) and a dry weight deviation (e.g. 0.132 pg).

The modeling environment in agent-based model(s) module 316 may be discretized at the molecular scale and the initial concentration of molecular fields may be assigned to each grid cell. Molecular species (e.g., metabolites) may be modeled using ordinary differential equations (ODEs) and allowed to diffuse between boxes with the diffusion of molecules governed by Fick's Second Law:

$$\frac{\partial [ C ]}{\partial T} = D ( \frac{{\partial }^{2} [ C ]}{\partial {x}^{2}} + \frac{{\partial }^{2} [ C ]}{\partial {y}^{2}} + \frac{{\partial }^{2} [ C ]}{\partial {z}^{2}} ) .$$

Diffusion may be modeled using the algorithm proposed by Grajdeanu. Based on this algorithm the concentration in each grid cell depends on the concentration in neighboring grid cells, the distance between cells, and the diffusion coefficient, which may be calculated according to:

$${d}_{j} = \vert {x}_{i} - {x}_{i}^{j} \vert ,$$

$$C ( {x}_{i} ) = C ( {x}_{i} ) + A {\sum }_{j = 1}^{n} ( C ( {x}_{i}^{j} ) - C ( {x}_{i} ) ) {e}^{- \frac{{d}_{j}^{2}}{D}} ,$$

$$and$$

$$A {\sum }_{j = 1}^{n} {e}^{- {d}_{j}^{2} / D} = 1 .$$

The movement of agents (representing microbes) may be modeled by random walk (suggested for time steps greater than 30 minutes) or biophysical flagellar movement, such as running and tumbling. A pairwise collision force may be applied to all overlapping microorganisms to avoid collision of diffusing bacterial agents. The magnitude of this force is proportional to the log of the ratio of the distance between two bacteria centers and the sum of their radii.

The agent-based model(s) module 316 then runs the simulation, also referred to herein as the in silico experiment, for the desired number of time steps. At each time step, a range of data may be stored such as coordinates of microorganisms, cell population, and the concentration of molecular fields. Microorganisms may be represented by autonomous agents possessing cellular characteristics including growth, division, and migration.

Microorganism growth, death, and division rules and rates may be naturally calculated from metabolic interactions or implemented based on experimental studies of morphogenesis in individual bacteria. In addition to characteristics of agents, agent-based model tools may provide other aspects of the simulation such as environmental boundaries, physical factors (e.g., crowding and steric repulsion), and collision detection.

The flux balance analysis module 318 uses flux balance analysis to predict metabolic interactions of microorganisms with the environment, and hence, identify their microbial growth. The flux balance analysis module 318 calculates the flow of metabolites through biochemical reactions in a metabolic network. The fluxes may be computed by optimizing an objective function

Z=c^{T}v,

where v is the vector of target fluxes. The linear programming problem is therefore to solve:

S·v=0,

where S is anm×n stoichiometric matrix of biochemical reactions with m compounds and n reactions, subject to lower and upper bounds for the vector v and a linear combination of fluxes Z as the objective function. Each agent may be assigned its metabolic models according to agent type. A linear programming (LP) solver such as GLPK (GNU Linear Programming Kit) or COIN-OR Linear Programming (CLP) may be used to solve LP problems for FBA. Lower bounds of fluxes may be updated according to the local concentration of metabolites in the vicinity of the microorganism. At each time step, LP solver may solve LP problems for each microorganism and updates environmental concentrations of the metabolites that are involved in exchange metabolic interactions.

Additionally, the biomass accumulated by an individual agent may be updated according to an exponential growth model using the optimal biomass flux computed by FBA

$${Biomass}_{t + 1} = {Biomass}_{t} + {v}_{biomass} \times {Biomass}_{t} \times dt .$$

Once accumulated biomass reaches a maximal dry weight (e.g. 1.172 pg), microbes replicate. When the accumulated biomass drops below a minimal dry weight (e.g. 0.083 pg), microorganisms die.

At each time step, molecular fields may be evaluated and field concentrations may be updated according to metabolic interactions of host gut microbiota and microbial strains in the microbiome therapeutic.

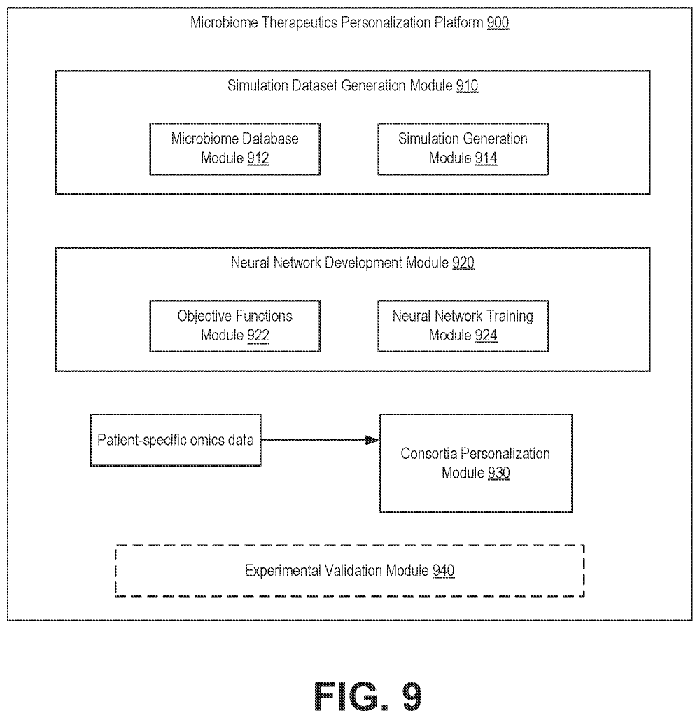

FIG. 9 shows a block diagram illustrating an example module architecture for a microbiome therapeutic personalization platform 900 for personalization of microbiome therapeutics for various diseases, according to an embodiment. The computational platform 900 may be implemented as the computational platform 106 in the computing system 100, as an illustrative and non-limiting example. It should be appreciated that the modules of the computational platform 900 are exemplary and non-limiting, and that the computational platform 900 may be implemented with other modules and sub-modules without departing from the scope of the present disclosure.

The computational platform 900 comprises a plurality of modules, including a simulation dataset generation module 910 configured to build a dataset of simulations for training of a neural network, a neural network development module 920, a consortia personalization module 930 configured to personalize the abundance levels of the target microbiome therapeutic to improve its therapeutic effects, and optionally an experimental validation module 940 configured to validate modules of the computing platform 900 based on experimental data.

The simulation dataset generation module 910 may simulate the interactions between a variety of microbiome samples with a variety of compositions of the target microbiome therapeutic. Various data types may be collected from these simulations and may be stored in data-holding subsystem 104 for future use. As illustrative and non-limiting examples, abundance of microbial taxa and concentration of different metabolites during or at the end of simulations may be stored.

The simulation dataset generation module 910 may comprise a microbiome database module 912 configured to store omics data, including one or more of metagenomic, metatranscriptomic, metaproteomic, or metabolomic data, from a set of microbiome samples from patients with a certain disease. As an illustrative and non-limiting example, the microbiome database module could include at least 100 metagenomic samples from patients with Type 2 Diabetes to capture a wide microbiome inter-individual variability.

The simulation dataset generation module may further comprise a simulation generation module 914 configured to generate simulations of a plurality of microbiome samples stored in microbiome database module 912 in interaction with a plurality of the target microbiome therapeutic variation in abundance level of its strains for a certain amount of simulation time.

The simulations may be conducted using the microbiome therapeutic modeling platform 300. As an illustrative and non-limiting example, 5,000 random compositions of the target microbiome therapeutic may be created. For example, random compositions may be created by randomly choosing a cell count, relative abundance, or absolute abundance for each of the strains. Additionally, for example, 100 microbiome samples from the microbiome database module 912 may be used to form 500,000 simulations.

It will be appreciated that the neural network development module 920 may have similar specifications as the neural network development module 214, as discussed above.

It will be appreciated that neural network development module 920 may be replaced with a machine learning development module 920 configured to train one or more machine learning models to predict final microbiome composition or certain microbiome features based on initial microbiome composition interacting with microbial consortia using the data generated in simulation dataset generation module 910 without departing from the scope of the present disclosure.

The consortia personalization module 930 is configured to personalize strain cell count, relative abundance, or absolute abundance for the target microbial consortia according to a patient's baseline microbiome features. In strain selection module 210, a nominal cell count or relative may be used for all the strains. In the consortia optimization module 220, the cell counts or relative abundances may be improved to according to objective functions determined in objective functions module 214-A using a single-objective or multi-objective optimization algorithm.

It will be appreciated that the experimental validation module 940 may have similar specifications as the experimental validation module 230, as discussed above.

To demonstrate the accuracy and advantages of the systems and methods provided herein relative to previous approaches, the results of multiple modeling and analysis studies that are illustrated in FIGS. 10-12. First, fifteen different human gut microbiome samples were simulated in the absence of microbiome therapeutics to demonstrate the ability to accurately represent the gut microbiome. Second, microbiome dynamics were predicted in a mouse model in the absence and presence of a microbiome therapeutic. Third, microbiome dynamics in interaction with a microbiome therapeutic were predicted in a mouse model. Fourth, design of enhanced microbiome therapeutics was evaluated using a mouse model.

For each simulated microbiome sample, it is expected that, in the absence of external stimuli, the composition of the microbiome over the time of simulation would not deviate from its initial composition.

FIG. 10 depicts a set of graphs 1000 illustrating results for twenty-four hours in silico experiments for fifteen human fecal samples, including 10 samples from a study of early onset Crohn's disease (CD) patients and 5 samples from a cohort of individuals with allergic diseases who participated in a Phase I clinical trial. For the CD study, paired-end Illumina raw reads for five healthy controls and five CD patients were retrieved from NCBI SRA under the accession SRP057027. For allergic patients, paired-end Illumina raw reads were provided by Siolta Therapeutics, Inc. Raw reads underwent pre-processing and analysis using the microbiome characterization module 312. Each microbiome was constructed using the agent-based model(s) module 316 and simulated for 24 hours with a time step of one hour. Alpha diversity and Aitchison distance were monitored throughout the simulation. Alpha diversity was calculated using the Shannon diversity index, and is depicted in the graph 1005. Aitchison distance was calculated by taking the Euclidean distance between the centered-log transformed samples, and is depicted in the graph 1010. FIG. 10 depicts that all the microbiome samples show a change of <10% in Shannon index throughout the simulation and an Aitchison distance of <20 between the final and initial composition of the simulated microbiome, confirming that the complex, multiscale dynamics of the human gut microbiota is captured over time.

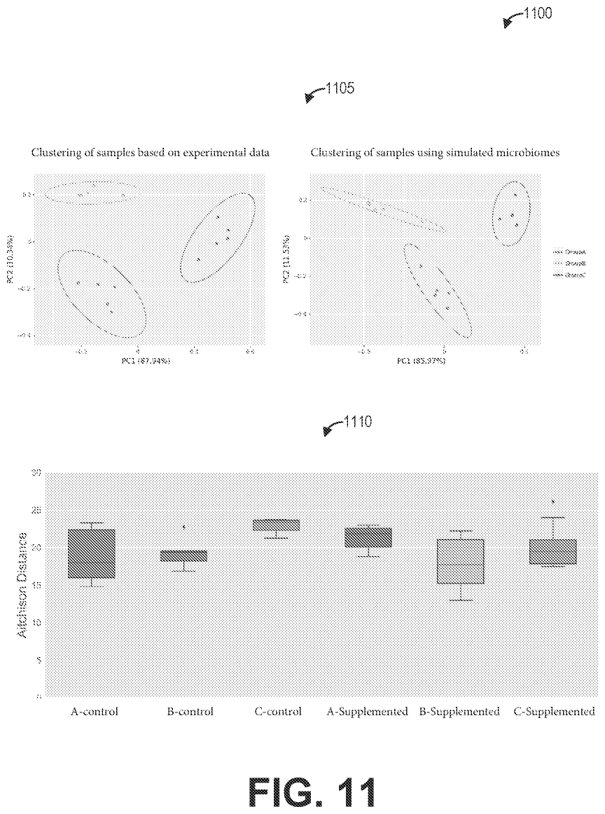

FIG. 11 depicts a set of graphs 1100 illustrating results for an animal study to demonstrate the dynamics of the metabolic interactions between microorganisms in a microbiome are accurately captured. Taxonomic data obtained from mice fecal samples were used to build subject-specific models of the gut microbiome for each mouse in this group (total of 5 for each group). Each sample was simulated for 7 days with a time step of 1 hour (total of 168 hours) to replicate the experiments.

Graph 1105 depicts that, using Principal Coordinate Analysis (PCoA), it could be seen that mice from each microbiota background will cluster together when we used taxonomic data obtained from fecal samples after 7 days. Similarly, simulated compositions (final composition after a 7-day simulation) also resulted in similar clustering of mice from different microbiota backgrounds.

Graph 1110 shows the compositional difference between experimental and simulated microbiome compositions using Aitchison distance across three groups of mice (A, B, and C) with distinct baseline microbiota compositions. An Aitchison distance of <25 is representative of closely related microbiome compositions between two samples. Graph 1110 shows the Aitchison distance for all the 15 simulated samples from control groups (absence of microbiome therapeutic) is <25, demonstrating that the simulated control microbiomes are closely related in composition to the experimental data. These comparisons confirm that the platform accurately captures the time-dependent characteristics of the community of microorganisms in the gut microbiome in the absence of external stimuli.

Graph 1110 further shows the compositional difference between experimental and simulated microbiome compositions interacting with a microbiome therapeutic using Aitchison distance. Again, taxonomic data obtained from mice fecal samples were used to build subject-specific models of the gut microbiome for each mouse in the supplemented group (total of 25 mice). The initial relative abundance of strains of the microbiome therapeutic was calibrated with a trial-and-error process. Each sample was simulated for 7 days with a time step of 1 hour (total of 168 hours) to replicate the experiments.

Graph 1110 shows the Aitchison distance for all the 25 simulated samples from the supplemented group is <25, confirming that the composition of simulated microbiomes in the presence of a microbiome therapeutic is significantly similar to experimentally-obtained microbiome compositions.

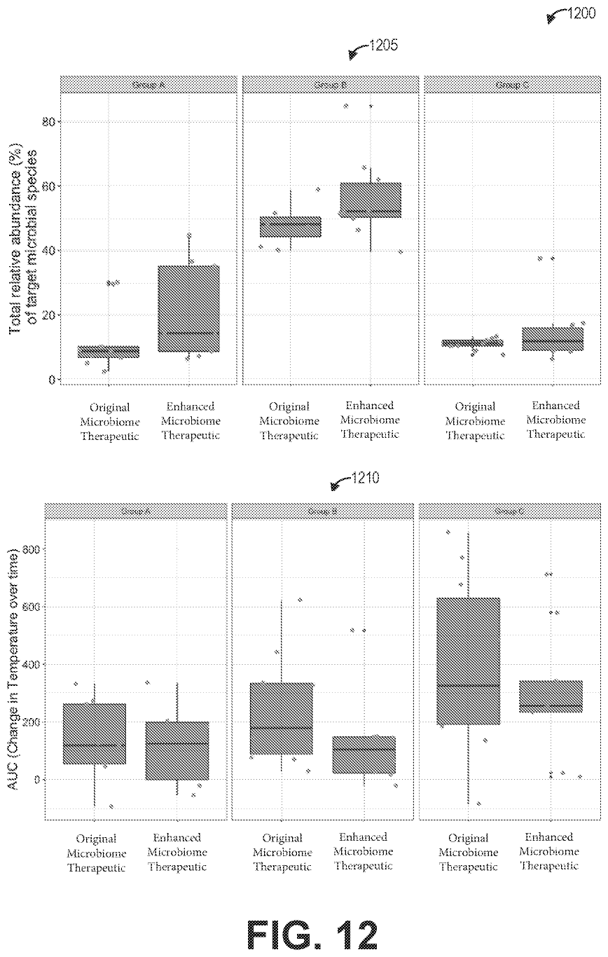

FIG. 12 depicts a set of graphs 1200 illustrating results for an animal study to demonstrate enhanced effectiveness of designed microbiome therapeutics. Mice were treated with an original multi-strain microbiome therapeutic and enhanced microbiome therapeutics were predicted using the platform outlined in this disclosure. The objective functions were identified as maximizing the total relative abundance of a set of microbial classes and genera including Clostridia, Lactobacillus, and Bifidobacteria. Enhanced microbiome therapeutics were predicted for three groups of mice with distinct baseline microbiota compositions. A subject-specific model of the gut microbiome was built for each mouse microbiome.

Graph 1205 shows the improvement in gut microbiome composition (higher relative abundance of target species) as a result of designing more effective compositions for each background gut microbiota. The average total relative abundance of target species was increased by 83% for Group A, 17% for Group B, and 29% for Group C.

Additionally, changes in immune response as a result of using enhanced microbiome therapeutics for each microbiota background compared to the original microbiome therapeutics compositions were evaluated. Plasma levels of MCPT-1 and IgE were shown to be either maintained or significantly reduced (up to 22%), indicating an effective immune response.

Graph 1210 depicts body temperature drop from the baseline temperature, as one of the primary factors that characterizes host response, with a lower area under the curve (AUC) of core body temperature change from the baseline representing a more effective immune response. Our results show that the average AUC is reduced by 9% for Group A, 45% for Group B, and 23% for Group C.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods.

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, and so on, that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The previously described versions of the disclosed subject matter have many advantages that were either described or would be apparent to a person of ordinary skill. Even so, these advantages or features are not required in all versions of the disclosed apparatus, systems, or methods.

Additionally, this written description makes reference to particular features. It is to be understood that the disclosure in this specification includes all possible combinations of those particular features. Where a particular feature is disclosed in the context of a particular aspect or example, that feature can also be used, to the extent possible, in the context of other aspects and examples.

Also, when reference is made in this application to a method having two or more defined steps or operations, the defined steps or operations can be carried out in any order or simultaneously, unless the context excludes those possibilities.

Although specific examples of the invention have been illustrated and described for purposes of illustration, it will be understood that various modifications may be made without departing from the spirit and scope of the invention.

1. A method for high-throughput screening of microbiome therapeutic candidates for a medical condition, the method comprising:
identifying a plurality of microbiome features associated with the medical condition;
generating a plurality of simulations by modeling interactions between a plurality of microbiome therapeutic candidates with a plurality of microbiome samples associated with the medical condition; and
determining one or more microbiome therapeutic candidates from the plurality of microbiome therapeutic candidates that improve and/or modify one or more microbiome features from the plurality of microbiome features associated with the medical condition based on an outcome of the plurality of simulations.

2. The method of claim 1, where the plurality of microbiome features comprises microbial composition at one or more taxonomic levels, metabolite composition, microbial diversity, microbial gene content, microbial gene products, microbial pathways, or any combination thereof.

3. The method of claim 1, where the plurality of microbiome features is identified using one or more of metagenomics, metatranscriptomics, metaproeomics, or metabolomics data.

4. The method of claim 3, wherein the metagenomics, metatranscriptomics, metaproeomics, or metabolomics data are derived from public sources and/or from analysis of one or more microbiomes associated with the medical condition.

5. The method of claim 1, wherein the generating the plurality of simulations is carried out using three-dimensional individual-specific models of the plurality of microbiome samples using data comprising one or more of metagenomic data, metatranscriptomic data, metaproteomic data, and metabolomic data.

6. The method of claim 1, wherein the microbiome comprises a gut microbiome and each of the microbiome therapeutic candidates is a microbial consortia comprising one or more gut-associated microbial strains.

7. The method of claim 1, wherein the plurality of simulations is further expanded using a trained machine learning model using the plurality of simulations.

8. The method of claim 7, wherein the machine learning model is a trained artificial neural network such as a deep neural network.

9. The method of claim 7, wherein the trained machine learning model is configured to predict:
microbial composition at one or more taxonomic levels during or at the end of each of the plurality of simulations; and/or
one or more microbiome features associated with the medical condition during or at the end of each of the plurality of simulations.

10. The method of claim 1, wherein the determining is carried out by application of an optimization algorithm.

11. The method of claim 10, wherein the optimization algorithm is a multi-objective optimization algorithm.

12. The method of claim 14, wherein the optimizing or improving is carried out by application of an optimization algorithm.

13. The method of claim 12, wherein the optimization algorithm is a multi-objective optimization algorithm.

14. The method of claim 33, wherein the optimizing or improving comprises identifying the most effective cell count, relative abundance, or absolute abundance for one or more microbial strains in one or more microbiome therapeutic candidates.

15. A method for personalization of a microbiome therapeutic candidate for a medical condition, the method comprising:
identifying a plurality of microbiome features associated with disease the medical condition;
generating a plurality of simulations by modeling interactions between a plurality of varied compositions of a microbiome therapeutic candidate with a plurality of microbiome samples associated with the medical condition;
and
determining a personalized composition of a microbiome therapeutic candidate from the plurality of varied compositions of a microbiome therapeutic candidate according to a patient's baseline microbiome so as to improve and/or modify one or more microbiome features from the plurality of microbiome features associated with the medical condition.

16. The method of claim 15, where the plurality of microbiome features comprises microbial composition at one or more taxonomic levels, metabolite composition, microbial diversity, microbial gene content, microbial gene products, microbial pathways, or any combination thereof.

17. The method of claim 15, where the plurality of microbiome features is identified using one or more of metagenomics, metatranscriptomics, metaproeomics, or metabolomics data.

18. The method of claim 17, wherein the metagenomics, metatranscriptomics, metaproeomics, or metabolomics data are derived from public sources and/or from analysis of one or more patient microbiomes.

19. The method of claim 15, wherein the generating the plurality of simulations is carried out using three-dimensional individual-specific models of the plurality of microbiome samples using data comprising one or more of metagenomic data, metatranscriptomic data, metaproteomic data, and metabolomic data.

20. The method of claim 15, wherein the microbiome comprises a gut microbiome, the microbiome therapeutic candidate comprises one or more gut-associated microbial strains, and the gut microbiome is individualized to a subject prescribed the microbiome therapeutic candidate.

21. The method of claim 35, wherein the machine learning model is a trained artificial neural network such as a deep neural network.

22. The method of claim 35, wherein the trained machine learning model is configured to predict:
microbial composition at one or more taxonomic levels during or at the end of each of the plurality of simulations; and/or
one or more microbiome features associated with the medical condition during or at the end of each of the plurality of simulations.

23. The method of claim 15, wherein the personalized composition is personalized based at least in part on identification of the most effective cell count, relative abundance, or absolute abundance for one or more of the microbial strains comprising the microbiome therapeutic.

24. The method of claim 23, wherein the personalized composition is personalized with an optimization algorithm.

25. The method of claim 24, wherein the optimization algorithm is a multi-objective optimization algorithm.

26. A system comprising:
a processor; and
a non-transitory storage medium storing executable instructions that when executed cause the processor to:
generate a plurality of simulations by modeling interactions between a plurality of microbiome therapeutic candidates and a plurality of microbiome samples associated with a medical condition;
determine one or more microbiome therapeutic candidates, selected from the plurality of microbiome therapeutic candidates, that improve and/or modify one or more microbiome features associated with the medical condition based on an outcome of the plurality of simulations;
or
generate a plurality of simulations by modeling interactions between a plurality of varied compositions of a microbiome therapeutic candidate with a plurality of microbiome samples associated with the medical condition; and
determine a personalized composition of a microbiome therapeutic candidate from the plurality of varied compositions of a microbiome therapeutic candidate according to a patient's baseline microbiome so as to improve and/or modify one or more microbiome features from the plurality of microbiome features associated with the medical condition.

27. The system of claim 26, wherein, to generate a plurality of simulations by modeling the interactions between a plurality of microbiome therapeutic candidates with a plurality of microbiome samples, the non-transitory storage medium further stores executable instructions that when executed cause the processor to:
construct metabolic models for one or more microorganisms;
generate a three-dimensional individual-specific model of the microbiome using agent-based modeling integrated with metabolic models of one or more microorganisms; and
update, at each time step of a plurality of time steps, coordinates and biophysical characteristics of one or more microorganisms and concentrations of molecular fields corresponding to metabolites within the three-dimensional individual-specific model using flux balance analysis for each of the one or more microorganisms to predict their growth and replication.

28. The system of claim 26, wherein, to determine one or more microbiome therapeutic candidates that improve and/or modify one or more microbiome features from the plurality of microbiome features associated with the medcial condition from the plurality of simulations, the non-transitory storage medium further stores executable instructions that when executed cause the processor to run a single-objective or multi-objective optimization algorithm including but not limited to:
create an initial random population of potential microbiome therapeutic candidates;
evaluate one or more microbiome features from the plurality of microbiome features for microbiome therapeutic candidates in this initial population;
rank microbiome therapeutic candidates according to evaluation;
create a subsequent population of microbiome therapeutic candidates using selection and transformation;
combine the two populations of microbiome therapeutic candidates; and
repeat this process for two or more generations.

29. The system of claim 26, wherein, to determine a personalized composition of a microbiome therapeutic candidate from the plurality of varied compositions of a microbiome therapeutic candidate according to a patient's baseline microbiome so as to improve and/or modify one or more microbiome features from the plurality of microbiome features associated with the medical condition, the non-transitory storage medium further stores executable instructions that when executed cause the processor to run a single-objective or multi-objective optimization algorithm including but not limited to:
create an initial random population of microbiome therapeutic candidate compositions;
evaluate one or more microbiome features from the plurality of microbiome features for microbiome therapeutic candidate compositions in this initial population;
rank microbiome therapeutic candidate compositions according to evaluation;
create a subsequent population of microbiome therapeutic candidate compositions using selection and transformation;
combine the two populations of microbiome therapeutic candidate compositions; and
repeat this process for two or more generations.

30. A system comprising:
a processor; and
a non-transitory storage medium storing executable instructions that when executed cause the processor to carry out the method of claim 1.

31. A system comprising:
a processor; and
a non-transitory storage medium storing executable instructions that when executed cause the processor to carry out the method of claim 15.

32. The method of claim 1, wherein the medical condition is a disease.

33. The method of claim 1, further comprising:
optimizing or improving a composition of the one or more microbiome therapeutic candidates to further improve and/or modify the one or more microbiome features from the plurality of microbiome features associated with the medical condition.

34. The method of claim 15, wherein the medical condition is a disease.

35. The method of claim 15, wherein the plurality of simulations is further expanded using a trained machine learning model using the plurality of simulations.

36. The system of claim 27, wherein the non-transitory storage medium further stores executable instructions that when executed cause the processor to:
train a machine learning model using plurality of simulations; and
expand the plurality of simulations using the trained machine learning model by generating more simulations outside of the plurality of simulations.
